(19) United States
(12) Patent Application Publication
(10) Pub. No.: US 2015/0320374 A1
(43) Pub. Date: Nov. 12, 2015

(54) DIAGNOSTIC X-RAY APPARATUS AND DIAGNOSTIC ULTRASOUND APPARATUS

(71) Applicants: Kabushiki Kaisha Toshiba, Minato-ku (JP); Toshiba Medical Systems Corporation, Otawara-shi (JP)

(72) Inventors: Hisayuki UEHARA, Otawara (JP); Masaki AKIYAMA, Otawara (JP); Hajime YOSHIDA, Nasushiobara (JP)

(73) Assignees: Kabushiki Kaisha Toshiba, Minato-ku (JP); Toshiba Medical Systems Corporation, Otawara-shi (JP)

(21) Appl. No.: 14/805,054

(22) Filed: Jul. 21, 2015

Related U.S. Application Data
(63) Continuation of application No. PCT/JP2014/051147, filed on Jan. 21, 2014.

(30) Foreign Application Priority Data
Jan. 22, 2013 (JP) 2013-009410
Jan. 21, 2014 (JP) 2014-008913

(51) Int. Cl. A61B 6/00; A61B 8/00; A61B 8/08; A61B 6/12; A61B 8/06

(52) U.S. Cl. CPC A61B 6/4417; A61B 6/12; A61B 8/06; A61B 6/463; A61B 8/5207; A61B 8/4444; A61B 8/4245

(57) ABSTRACT

A diagnostic X-ray apparatus of an embodiment includes determining circuitry, projection image generating circuitry, and a display. The determining circuitry determines, based on position information of a probe of a diagnostic ultrasound apparatus acquired from the diagnostic ultrasound apparatus, at least one of an angle horizontal to a scan plane of the probe and an angle vertical to the scan plane of the probe to be an X-ray irradiation direction in processing to perform puncture using a puncture needle. The projection image generating circuitry generates, based on the position information and the X-ray irradiation direction, projection image data that projects an ultrasound image of the scan plane in the X-ray irradiation direction. The display displays X-ray image data containing the puncture needle photographed in the X-ray irradiation direction and the projection image data generated by the projection image generating circuitry.

### CROSS-REFERENCE TO RELATED APPLICATIONS

This application is a continuation of PCT international application Ser. No. PCT/JP2014/051147 filed on Jan. 21, 2014 which designates the United States, incorporated herein by reference, and which claims the benefit of priority from Japanese Patent Application No. 2013-009410, filed on Jan. 22, 2013 and Japanese Patent Application No. 2014-008913, filed on Jan. 21, 2014, the entire contents of which are incorporated herein by reference.

### FIELD

Embodiments described herein relate generally to a Diagnostic X-ray apparatus and a diagnostic ultrasound apparatus.

### BACKGROUND

Diagnostic ultrasound apparatuses have been conventionally generally used at the time of puncture processing in examinations that collect tissues such as tumors or treatments that locally administer medicines. A manipulator (hereinafter, referred to as an “operator”) such as a doctor, for example, advances a puncture needle to a region to be punctured while viewing an image of the puncture needle in an ultrasound image captured by a diagnostic ultrasound apparatus.

### BRIEF DESCRIPTION OF THE DRAWINGS

FIG. 1 is a diagram illustrating a configuration example of a puncture supporting system according to an embodiment;

FIG. 2 is a functional block diagram illustrating configuration examples of a diagnostic ultrasound apparatus according to the embodiment and a diagnostic X-ray apparatus according to the embodiment;

FIG. 3 is a diagram illustrating examples of a scan plane of the diagnostic ultrasound apparatus and an X-ray irradiation direction by the diagnostic X-ray apparatus;

FIG. 4 is a diagram illustrating an example of an irradiation range when X-rays are applied in the horizontal direction relative to a scan plane;

FIG. 5 is a diagram illustrating an example of the irradiation range when X-rays are applied in the vertical direction relative to the scan plane;

FIG. 6A is a diagram illustrating an example of an operation to generate projection image data by data correcting circuitry according to the embodiment;

FIG. 6B is a diagram illustrating an example of the operation to generate projection image data by the data correcting circuitry according to the embodiment;

FIG. 6C is a diagram illustrating an example of the operation to generate projection image data by the data correcting circuitry according to the embodiment;

FIG. 7 is a diagram illustrating an example of an image generated by image processing circuitry according to the embodiment;

FIG. 8 is a diagram illustrating an example of superimposed image data generated by the image processing circuitry according to the embodiment;

FIG. 9 is a flowchart illustrating a processing procedure by the diagnostic ultrasound apparatus according to the embodiment;

FIG. 10 is a flowchart illustrating a processing procedure by the diagnostic X-ray apparatus according to the embodiment;

FIG. 11A is a diagram illustrating an example of a relation between a puncture needle and a region to be punctured in a scan plane in the puncture supporting system according to the embodiment;

FIG. 11B is a diagram illustrating an example of the relation between the puncture needle and the region to be punctured in the scan plane in the puncture supporting system according to the embodiment;

FIG. 11C is a diagram illustrating an example of the relation between the puncture needle and the region to be punctured in the scan plane in the puncture supporting system according to the embodiment;

FIG. 12A is a diagram illustrating an example of the relation between the puncture needle and the region to be punctured in the scan plane in the puncture supporting system according to the embodiment;

FIG. 12B is a diagram illustrating an example of the relation between the puncture needle and the region to be punctured in the scan plane in the puncture supporting system according to the embodiment;

FIG. 12C is a diagram illustrating an example of the relation between the puncture needle and the region to be punctured in the scan plane in the puncture supporting system according to the embodiment;

FIG. 13A is a diagram illustrating an example of the relation between the puncture needle and the region to be punctured in the scan plane in the puncture supporting system according to the embodiment;

FIG. 13B is a diagram illustrating an example of the relation between the puncture needle and the region to be punctured in the scan plane in the puncture supporting system according to the embodiment;

FIG. 13C is a diagram illustrating an example of the relation between the puncture needle and the region to be punctured in the scan plane in the puncture supporting system according to the embodiment;

FIG. 14 is a diagram illustrating an example of modifications of superimposed image data generated when X-rays are applied in the horizontal direction relative to a scan plane of the diagnostic ultrasound apparatus; and

FIG. 15 is a diagram illustrating an example of the modifications of the superimposed image data generated when X-rays are applied in the vertical direction relative to the scan plane of the diagnostic ultrasound apparatus.

### DETAILED DESCRIPTION

A diagnostic X-ray apparatus of an embodiment includes determining circuitry, projection image generating circuitry, and a display. The determining circuitry determines, based on position information of a probe of a diagnostic ultrasound apparatus acquired from the diagnostic ultrasound apparatus, at least one of an angle horizontal to a scan plane of the probe and an angle vertical to the scan plane of the probe to be an X-ray irradiation direction in processing to perform puncture using a puncture needle. The projection image generating circuitry generates, based on the position information and the X-ray irradiation direction, projection image data that projects an ultrasound image of the scan plane in the X-ray irradiation direction. The display displays X-ray image data containing the puncture needle photographed in the X-ray irradiation direction and the projection image data generated by the projection image generating circuitry.

The following describes a diagnostic X-ray apparatus and a diagnostic ultrasound apparatus according to an embodiment with reference to the drawings.

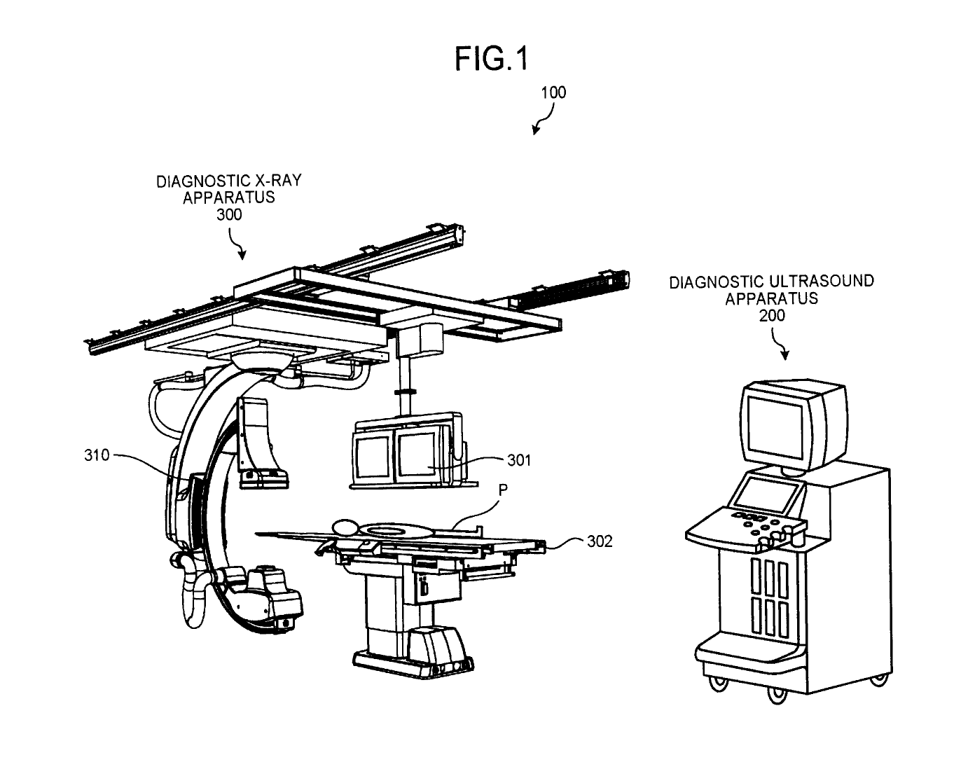

The embodiment describes a puncture supporting system 100 including a diagnostic X-ray apparatus as an example. FIG. 1 is a diagram illustrating a configuration example of the puncture supporting system 100 according to the embodiment. As illustrated in FIG. 1, the puncture supporting system 100 includes a diagnostic ultrasound apparatus 200 and a diagnostic X-ray apparatus 300. The diagnostic ultrasound apparatus 200 include a probe (not illustrated). The probe may be fitted with a puncture needle insertion guide. The diagnostic X-ray apparatus 300 includes a display 301, a table 302, and a C-arm 310. A subject P illustrated in FIG. 1 is not included in the puncture supporting system 100. A position in the puncture supporting system 100 is defined by an X-Y-Z coordinate system. Detailed configurations of the diagnostic ultrasound apparatus 200 and the diagnostic X-ray apparatus 300 will be described below.

The puncture supporting system 100 performs puncture processing using the diagnostic ultrasound apparatus 200 on the subject P. A manipulator (hereinafter, referred to as an “operator”) such as a doctor, for example, determines a region to be punctured by referring to an ultrasound image captured by the diagnostic ultrasound apparatus 200. The operator, for example, performs examinations that collect tissues such as tumors with a puncture needle, treatments that locally administer medicines from the tip of a puncture needle, thermocauterectomy that applies microwaves or radio waves from the tip of a puncture needle, or the like while referring to the ultrasound image of the region to be punctured captured by the diagnostic ultrasound apparatus 200.

During such puncture processing, the puncture needle is advanced along a scan plane of a diagnostic ultrasound apparatus. However, the puncture needle may deviate from the scan plane during the puncture processing. The operator cannot clearly determine whether the puncture needle viewed as an ultrasound image is its tip or its central part. Given this situation, it may not be checked by conventional diagnostic ultrasound apparatuses whether the puncture needle has reached a region to be punctured. In view of such circumstances, in the puncture supporting system 100 according to the embodiment, the diagnostic X-ray apparatus 300, based on position information of a scan plane of the probe of the diagnostic ultrasound apparatus 200 acquired from the diagnostic ultrasound apparatus 200 that performs puncture using a puncture needle, determines an X-ray irradiation direction that crosses the scan plane by a certain angle. The diagnostic X-ray apparatus 300 generates projection image data that projects the scan plane in the X-ray irradiation direction based on the position information and the X-ray irradiation direction. The diagnostic X-ray apparatus 300 generates a superimposed image that superimposes an X-ray image containing the puncture needle captured in the X-ray irradiation direction and a projection image.

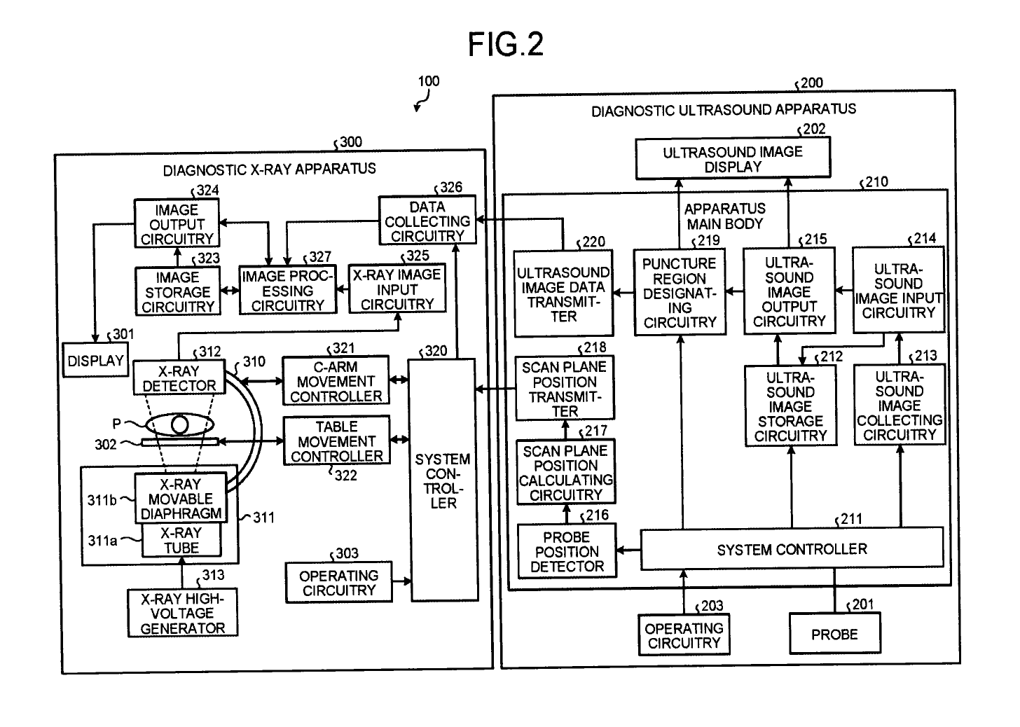

Described with reference to FIG. 2 are detailed configurations of the diagnostic ultrasound apparatus 200 and the diagnostic X-ray apparatus 300. FIG. 2 is a functional block diagram illustrating configuration examples of the diagnostic ultrasound apparatus 200 according to the embodiment and the diagnostic X-ray apparatus 300 according to the embodiment.

As illustrated in FIG. 2, the diagnostic ultrasound apparatus 200 includes a probe 201, an ultrasound image display 202, operating circuitry 203, and an apparatus main body 210. The ultrasound probe 201 is connected to the apparatus main body 210 to perform the transmission and reception of ultrasound waves. The ultrasound probe 201 includes, for example, a plurality of piezoelectric transducer elements. The piezoelectric transducer elements generate ultrasound waves based on drive signals supplied from a system controller 211 of the apparatus main body 210 described below, receive reflected waves from the subject P, and convert them into electric signals. The ultrasound probe 201 includes a matching layer provided on the piezoelectric transducer elements and a backing member that prevents ultrasound waves from transmitting backward from the piezoelectric transducer elements.

When ultrasound waves are transmitted to the subject P by the ultrasound probe 201, the transmitted ultrasound waves are successively reflected by discontinuity surfaces of acoustic impedance in the body tissue of the subject P and are received by the piezoelectric transducer elements of the ultrasound probe 201 as reflected wave signals. The amplitude of the reflected wave signals depends on a difference in acoustic impedance on the discontinuity surfaces by which the ultrasound waves are reflected. The reflected wave signals when a transmitted ultrasound pulse is reflected by moving bloodstreams or surfaces such as heart walls are subjected to frequency shift depending on velocity components of moving bodies relative to an ultrasound transmission direction by the Doppler effect.

The ultrasound image display 202 is a display device such as a monitor that displays ultrasound image data. The operating circuitry 203 transfers various setting requests received from the operator to the apparatus main body 210. The operating circuitry 203, for example, receives the designation of a photographing mode from the operator. This operation causes ultrasound image input circuitry 214 to generate the ultrasound image data with the designated photographing mode. The photographing mode includes “B mode,” which photographs B mode images, “M mode,” which photographs M mode images, “C mode,” which photographs color Doppler images, and “D mode,” which photographs Doppler waveform images. The operating circuitry 203, for example, receives an instruction to collect an ultrasound image from the operator. This operation causes the system controller 211 to transmit ultrasound waves to the subject P. The operating circuitry 203, for example, receives the designation of a region to be punctured from the operator. This operation causes puncture region designating circuitry 219 to generate image data that superimposes an area indicating the region to be punctured on the ultrasound image.

The apparatus main body 210 includes the system controller 211, ultrasound image storage circuitry 212, ultrasound image collecting circuitry 213, the ultrasound image input circuitry 214, ultrasound image output circuitry 215, a probe position detector 216, scan plane position calculating circuitry 217, a scan plane position transmitter 218, the puncture region designating circuitry 219, and an ultrasound image data transmitter 220.

The system controller 211 includes a trigger generating circuit, a transmission delay circuit, and a pulser circuit and supplies drive signals to the ultrasound probe 201. The pulser circuit repeatedly generates rate pulses for forming transmission ultrasound waves at a certain rate frequency. The transmission delay circuit gives transmission delay times for the respective piezoelectric transducer elements used for focusing the ultrasound waves generated by the ultrasound probe 201 into a beam shape and determining transmission directivity to the respective rate pulses generated by the pulser circuit. The trigger generating circuit supplies drive signals to the ultrasound probe 201 with timing based on the rate pulses.

The ultrasound image storage circuitry 212 stores therein the ultrasound image data generated by the ultrasound image input circuitry 214.

The ultrasound image collecting circuitry 213 includes an amplifier circuit, an A/D converter, an adder, and the like and performs various processing on the reflected wave signals received by the ultrasound probe 201 to generate reflected wave data. The amplifier circuit amplifies the reflected wave signals to perform gain correction processing. The A/D converter performs A/D conversion on the gain-corrected reflected wave signals to give a reception delay time necessary for determining reception directivity. The adder performs addition processing on the reflected wave signals processed by the A/D converter to generate the reflected wave data. The addition processing performed by the adder enhances a reflection component in a direction corresponding to the reception directivity of the reflected wave signals.

The ultrasound image input circuitry 214 generates the ultrasound image data from the reflected wave data generated by the ultrasound image collecting circuitry 213. The ultrasound image input circuitry 214, for example, receives the reflected wave data from the ultrasound image collecting circuitry 213 and performs logarithmic amplification, envelope demodulation processing, or the like thereon to generate data (B mode data) in which signal intensity is represented by the magnitude of brightness. The ultrasound image input circuitry 214 generates B mode image data from the B mode data. The ultrasound image input circuitry 214 performs frequency analysis on velocity information for the reflected image data received from the ultrasound image collecting circuitry 213, extracts bloodstreams, tissues, and contrast medium echo components by the Doppler effect, and generates data (Doppler data) that extracts moving body information such as average velocity, variance, and power for multiple points. The ultrasound image input circuitry 214 generates average velocity images, variance images, power images, or color Doppler image data as a combination of these images from the Doppler data. The ultrasound image input circuitry 214 generates M (motion) mode image data in a range gate set by a user from time-series data of the B mode data. The ultrasound image input circuitry 214 generates Doppler waveform image data obtained by plotting velocity information of bloodstreams or tissues in a range gate set. by the user in a time-series manner from time-series data of the Doppler data. The Doppler waveform image data is generated from Doppler data collected by the continuous wave (CW) Doppler method or the pulsed wave (PW) Doppler method.

The ultrasound image output circuitry 215 causes the ultrasound image display 202 to display the ultrasound image data from the ultrasound image input circuitry 214 or the ultrasound image data stored by the ultrasound image storage circuitry 212. The ultrasound image output circuitry 215, for example, causes the ultrasound image display 202 to display various ultrasound image data generated in various photographing modes in ultrasound examinations performed using the diagnostic ultrasound apparatus 200.

The probe position detector 216 determines a position of the probe 201. The probe position detector 216 determines the position of the probe 201 in the X-Y-Z coordinate system in the puncture supporting system 100. The probe position detector 216, for example, determines the position of the probe 201 using GPS. With the probe 201 attached to a certain position of the table, the probe position detector 216 may determine the position of the probe 201 in accordance with a movement amount from the certain position. The probe position detector 216 may determine the position of the probe 201 by receiving a signal from an ultrasound transmitter attached to the probe 201.

The scan plane position calculating circuitry 217 determines a position of the scan plane based on the position of the probe determined by the probe position detector 216. The scan plane position calculating circuitry 217, for example, determines the position of the scan plane based on a direction of the probe and the position of the probe. The scan plane position calculating circuitry 217 outputs the determined position of the scan plane as position information of the scan plane to the scan plane position transmitter 218. The position information of the scan plane is, for example, denoted as Σ (x, y, z) in the X-Y-Z coordinate system.

The scan plane position transmitter 218 transmits the position information of the scan plane acquired from the scan plane position calculating circuitry 217 to the diagnostic X-ray apparatus 300.

When the designation of the region to be punctured is received from the operator via the operating circuitry 203, the puncture region designating circuitry 219 generates image data that superimposes an area indicating the region to be punctured designated by the operator on the ultrasound image. The puncture region designating circuitry 219, for example, generates the image data that superimposes the region to be punctured shown by a rectangle or a circle on the ultrasound image data. The puncture region designating circuitry 219 outputs the generated ultrasound image data to the ultrasound image data transmitter 220. The puncture region designating circuitry 219 causes the ultrasound image display 202 to display the generated ultrasound image data.

The ultrasound image data transmitter 220 transmits the ultrasound image data generated by the puncture region designating circuitry 219 to the diagnostic X-ray apparatus 300.

As illustrated in FIG. 2, the diagnostic X-ray apparatus 300 includes the display 301, the table 302, operating circuitry 303, the C-arm 310, an X-ray high-voltage generator 313, a system controller 320, a C-arm movement controller 321, a table movement controller 322, image storage circuitry 323, image output circuitry 324, X-ray image input circuitry 325, data collecting circuitry 326, and image processing circuitry 327.

The display 301 displays, for example, X-ray images such as fluoroscopic images captured by the diagnostic X-ray apparatus 300. The table 302 mounts a subject P thereon. The table 302, for example, includes a tabletop that mounts the subject P thereon and is movable in the vertical direction and the horizontal direction. The table 302 can move the tabletop in the longitudinal direction or in both the longitudinal direction and the shorter side direction. The table 302 moves itself or the tabletop, thereby moving the subject P to a photographing area of the diagnostic X-ray apparatus 300.

The operating circuitry 303 is a control panel, a foot switch, a joystick, or the like and receives input of various operations to the diagnostic X-ray apparatus 300 from the operator. The operating circuitry 303, for example, receives an operation to be performed on the table 302 to move an object to be observed within the subject P to the center of a screen from the operator. This operation causes the table movement controller 322 to move the table 302 in accordance with the operation by the operator. The operating circuitry 303 receives an operation to rotate the C-arm 310 from the operator. This operation causes the C-arm movement controller 321 to rotate the C-arm 310 in accordance with the operation performed by the user.

The operating circuitry 303 receives settings of photographing conditions from the operator. The operating circuitry 303, for example, receives information such as source-image distance (SID) and field of view (FOV) from the operator. Values of SID and FOV may be held by the diagnostic X-ray apparatus 300 in advance. The operating circuitry 303 receives an instruction to collect X-ray image data from the operator.

The operating circuitry 303 receives a setting of the X-ray irradiation direction relative to the scan plane by the diagnostic ultrasound apparatus 200 from the operator. The operating circuitry 303, for example, receives a setting of the horizontal direction relative to the scan plane or the vertical direction relative to the scan plane. The operator can change the setting of the X-ray irradiation direction relative to the scan plane during scanning. The irradiation direction may be set at an angle relative to the scan plane.

The C-arm 310 supports an X-ray source 311 and an X-ray detector 312 so as to cause the two to face each other. The X-ray source 311 is a device that includes an X-ray tube 311a and an X-ray beam limiting device 311b and generates X-rays through a high voltage supplied from the X-ray high-voltage generator 313. The X-ray tube 311a applies X-rays. The X-ray beam limiting device 311b narrows the range of the X-rays applied from the X-ray tube 311a to the subject P to a range containing a region of interest of the subject P. The X-ray detector 312 is configured to detect the X-rays applied from the X-ray source 311 and having passed through the subject P. The pair of the X-ray source 311 and the X-ray detector 312 is configured to rotate around a geometrical rotational center (an isocenter).

The X-ray high-voltage generator 313 is a device that generates high voltage to be supplied to the X-ray source 311 and controls output of the X-rays applied from the X-ray tube 311 by controlling generated voltage and current.

The C-arm movement controller 321 controls the rotation and the like of the C-arm 310 under the control of the system controller 320. The C-arm movement controller 321, for example, rotates the C-arm 310 in the left anterior oblique view (LAO: the second oblique position) direction or the right anterior oblique view (RAO: the first oblique position) direction based on input signals from the operating circuitry 303.

The table movement controller 322 performs operation control of the table 302 under the control of the system controller 320. The table movement controller 322, for example, controls the movement of the table 302 in the vertical direction and the movement of the table 302 in the horizontal direction based on input signals from the operating circuitry 303.

The image storage circuitry 323 stores therein X-ray image data or the like. The image output circuitry 324 causes the display 301 to display the X-ray image data generated by the image processing circuitry 327. The X-ray image input circuitry 325 collects images that are the X-rays applied to the subject P and projected on the X-ray detector 312.

The system controller 320 performs the entire control of the diagnostic X-ray apparatus 300 based on instructions by the operating circuitry 303. When an instruction to photograph an X-ray image is received from the operator via the operating circuitry 303, for example, the system controller 320 controls the C-arm movement controller 321 and the table movement controller 322 to collect X-ray image data of the subject P.

The system controller 320 determines the X-ray irradiation direction that crosses the scan plane by a certain angle based on the position information of the scan plane of the probe 201 of the diagnostic ultrasound apparatus 200 acquired from the diagnostic ultrasound apparatus 200 that performs puncture using the puncture needle.

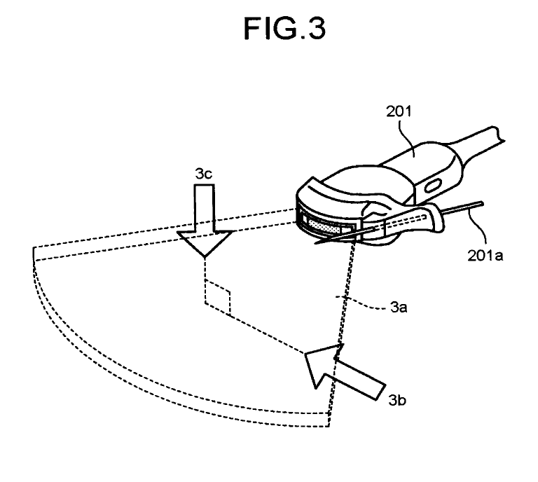

The system controller 320, for example, determines the X-ray irradiation direction that crosses the scan plane of the diagnostic ultrasound apparatus 200 by a certain angle received from the operator via the operating circuitry 303. The following describes a relation between the scan plane of the diagnostic ultrasound apparatus 200 and the X-ray irradiation direction by the diagnostic X-ray apparatus with reference to FIG. 3. FIG. 3 is a diagram illustrating examples of the scan plane of the diagnostic ultrasound apparatus 200 and the X-ray irradiation direction by the diagnostic X-ray apparatus 300. FIG. 3 illustrates an example of the scan plane of the ultrasound waves transmitted from the piezoelectric transducer elements of the ultrasound probe 201 and the reflected wave signals received by the piezoelectric transducer elements. scan plane 3a has a thickness corresponding to the width of the piezoelectric transducer elements.

When the X-ray irradiation direction relative to the scan plane 3a is set to be the horizontal direction, the diagnostic X-ray apparatus 300 applies X-rays from a direction indicated by an arrow 3b illustrated in FIG. 3. When the X-ray irradiation direction relative to the scan plane 3a is set to be the vertical direction, the diagnostic X-ray apparatus 300 applies X-rays from a direction indicated by an arrow 3c illustrated in FIG. 3.

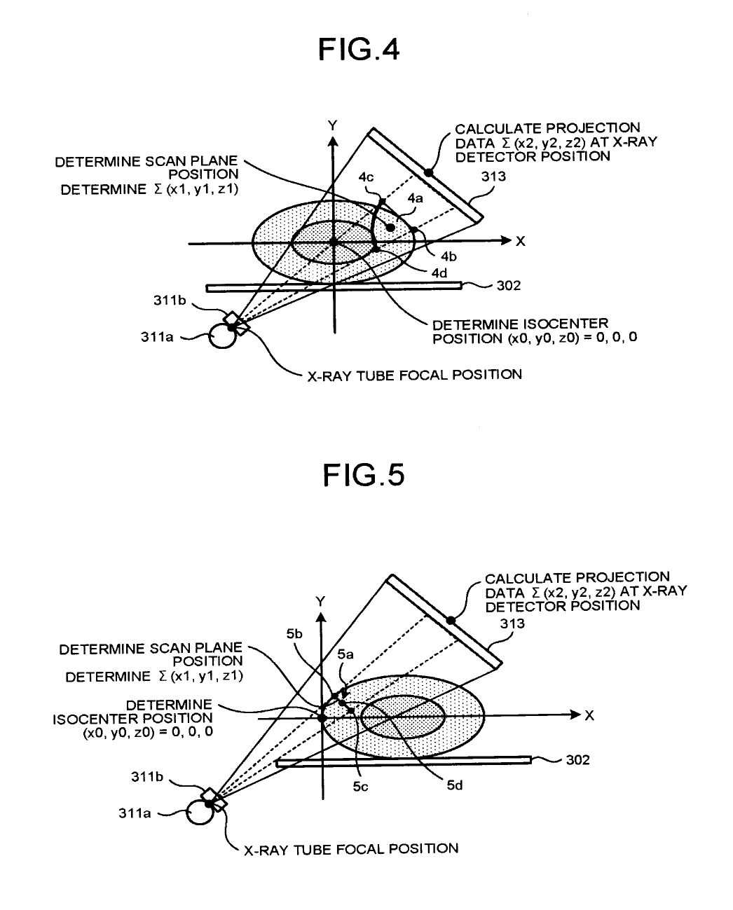

Described next with reference to FIG. 4 is an irradiation range when X-rays are applied in the horizontal direction relative to the scan plane of the diagnostic ultrasound apparatus 200. FIG. 4 is a diagram illustrating an example of the irradiation range when X-rays are applied in the horizontal direction relative to the scan plane. In other words, the certain angle is 0 degree in this example. In FIG. 4, the table 302 mounts the subject P thereon. FIG. 4 is a drawing when the subject P is viewed from the head side in the anteroposterior direction in an X-Y coordinate system.

First, the system controller 320 determines a position of the scan plane based on the position information of the scan plane acquired from the diagnostic ultrasound apparatus 200. The system controller 320, for example, determines the area 4a illustrated in FIG. 4 to be the position of the scan plane. In this example, the position of the scan plane 4a is denoted as Σ (x1, y1, z1).

When X-rays are applied in the horizontal direction relative to the scan plane, the system controller 320 rotates the C-arm to set the X-ray source 311 and the X-ray detector 312 at the positions illustrated in FIG. 4. Specifically, the system controller 320 determines an area containing points 4b, 4c, and 4d of the scan plane 4a to be the X-ray irradiation direction. A position of the scan plane 4a when the scan plane 4a is projected on the X-ray detector 312 is denoted as Σ (x2, y2, z2). Projection image data that projects the scan plane 4a will be described below.

Described next with reference to FIG. 5 is an irradiation range when X-rays are applied in the vertical direction relative to the scan plane. FIG. 5 is a diagram illustrating an example when X-rays are applied in the vertical direction relative to the scan plane. In other words, the certain angle is 90 degrees that vertically crosses the scan plane in this example. In FIG. 5, the table 302 mounts the subject P thereon. FIG. 5 is a drawing when the subject P is viewed from the head side in the anteroposterior direction in the X-Y coordinate system.

First, the system controller 320 determines a position of the scan plane based on the position information of the scan plane acquired from the diagnostic ultrasound apparatus 200. The system controller 320, for example, determines the area 5a illustrated in FIG. 5 to be the position of the scan plane. In this example, the position of the scan plane 5a is denoted as Σ (x1, y1, z1). The scan plane 5a illustrated in FIG. 5 is represented by a line, because the plane is along the Z-axis.

When X-rays are applied in the vertical direction relative to the scan plane 5a, the system controller 320 rotates the C-arm to set the X-ray source 311 and the X-ray detector 312 at the positions illustrated in FIG. 5. Specifically, the system controller 320 determines an area containing points 5b, 5c, and 5d of the scan plane 5a to be the X-ray irradiation direction. A position of the scan plane 5a when the scan plane 5a is projected on the X-ray detector 312 is denoted as Σ (x2, y2, z2). Projection image data that projects the scan plane 5a will be described below.

The system controller 320 outputs the position information of the scan plane to the data collecting circuitry 326. The system controller 320 photographs X-ray image data for the area determined to be the irradiation range.

Returning back to FIG. 2, the data collecting circuitry 326 generates projection image data that projects the scan plane in the irradiation range determined by the system controller 320. The data collecting circuitry 326, for example, generates the projection image data using the position information of the scan plane acquired from the system controller 320, the X-ray irradiation direction, and the ultrasound image data acquired from the diagnostic ultrasound apparatus 200.

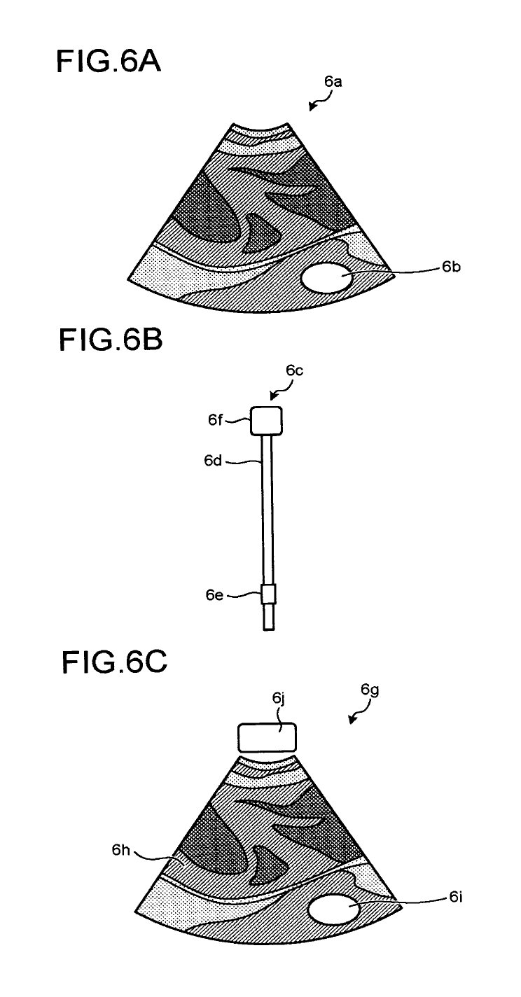

Described with reference to FIG. 6A to FIG. 6C is processing to generate the projection image data by the data collecting circuitry 326 according to the embodiment. FIG. 6A to FIG. 6C are diagrams illustrating an example of an operation to generate the projection image data by the data collecting circuitry 326 according to the embodiment. FIG. 6A is an ultrasound image 6a captured by the diagnostic ultrasound apparatus 200. A region 6b to be punctured is displayed in the ultrasound image 6a. When X-rays are applied in the horizontal direction relative to the ultrasound image 6a, the data collecting circuitry 326, for example, generates a projection image 6c illustrated in FIG. 6B. Specifically, the data collecting circuitry 326 generates the projection image 6c containing a scan plane 6d that projects the ultrasound image 6a, 6e schematically indicating a position at which the region 6b to be punctured is projected, and 6f schematically indicating the probe.

When X-rays are applied in the vertical direction relative to the ultrasound image 6a, the data collecting circuitry 326, for example, generates a projection image 6g illustrated in FIG. 6C. Specifically, the data collecting circuitry 326 generates the projection image 6g containing a scan plane 6h that projects the ultrasound image 6a, 6i schematically indicating a position at which the region 6b to be punctured is projected, and 6j schematically indicating the probe.

Returning back to FIG. 2, the image processing circuitry 327 generates superimposed image data that superimposes the X-ray image data containing the puncture needle captured in the X-ray irradiation direction determined by the system controller 320 and the projection image data generated by the data collecting circuitry 326.

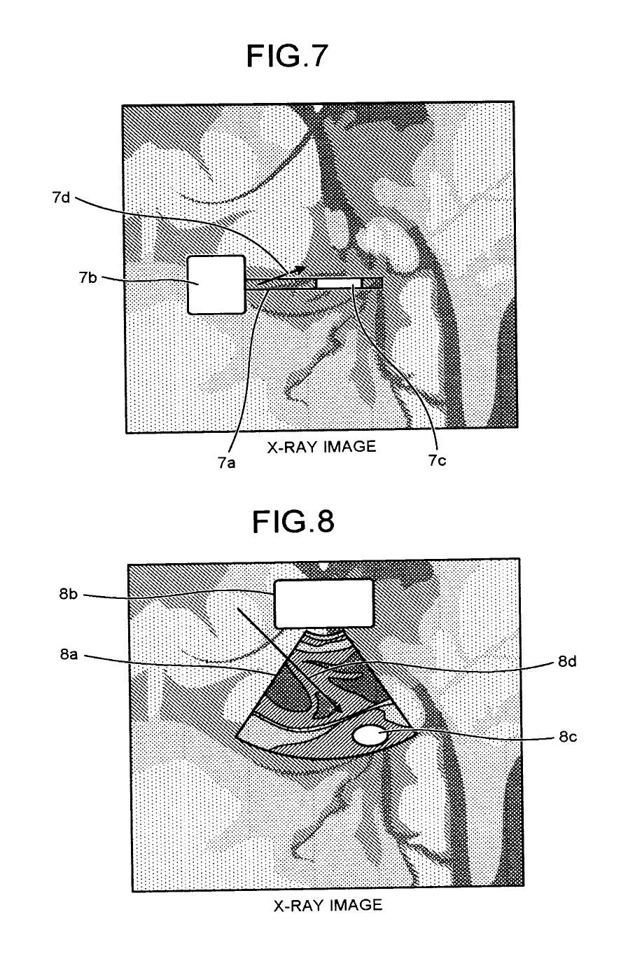

An example of the superimposed image data generated by the image processing circuitry 327 will be described with reference to FIG. 7 and FIG. 8. Described with reference to FIG. 7 is the superimposed image data generated when X-rays are applied in the horizontal direction relative to the scan plane. Described with reference to FIG. 8 is the superimposed image data generated when X-rays are applied in the vertical direction relative to the scan plane.

FIG. 7 is a diagram illustrating an example of an image generated by the image processing circuitry 327 according to the embodiment. As illustrated in FIG. 7, the image processing circuitry 327 generates the superimposed image data that superimposes the projection image data on the X-ray image data. Specifically, the image processing circuitry 327 superimposes the projection image data containing information 7a schematically indicating the scan plane, information 7b schematically indicating the probe, and information 7c schematically indicating the region to be punctured on the X-ray image data. A puncture needle 7d is projected on the X-ray image data. With this processing, the operator can check whether the puncture needle does not deviate from the scan plane.

FIG. 8 is a diagram illustrating an example of the superimposed image data generated by the image processing circuitry 327 according to the embodiment. As illustrated in FIG. 8, the image processing circuitry 327 generates the superimposed image data that superimposes the projection image data on the X-ray image data. Specifically, the image processing circuitry 327 superimposes the projection image data containing an ultrasound image 8a, information 8b schematically indicating the probe, and information 8c schematically indicating the region to be punctured on the X-ray image data. A puncture needle 8d is projected on the X-ray image data. With this processing, the operator can check whether the puncture needle has reached the region to be punctured.

The image processing circuitry 327 outputs the generated X-ray image data to the image output circuitry 324. The image processing circuitry 327 stores the collected X-ray image data in the image storage circuitry 323.

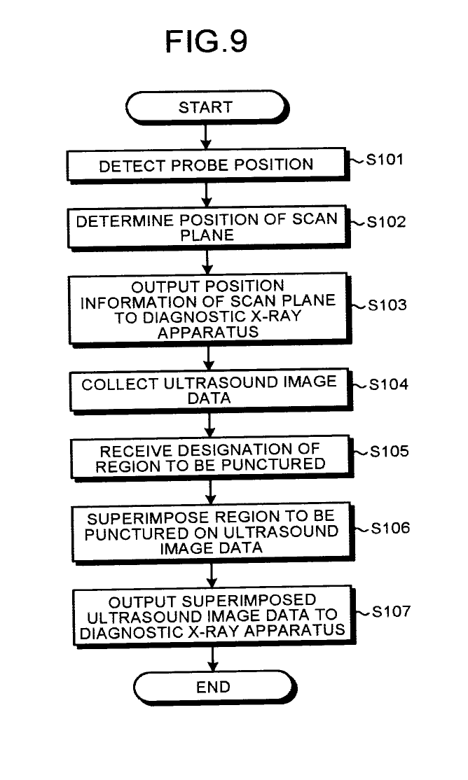

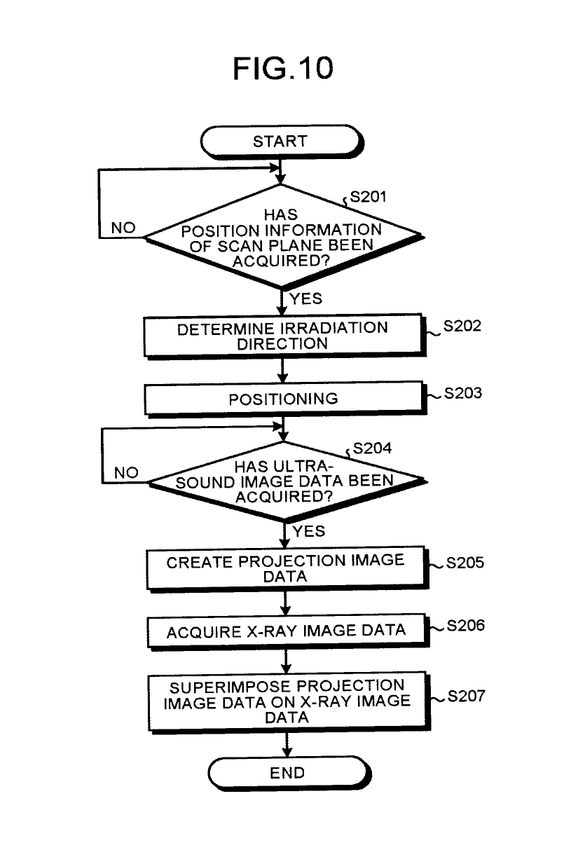

Next, a processing procedure by the puncture supporting system 100 will be described with reference to FIG. 9 and FIG. 10. Described with reference to FIG. 9 is a processing procedure by the diagnostic ultrasound apparatus 200. Described with reference to FIG. 10 is a processing procedure by the diagnostic X-ray apparatus 300. FIG. 9 is a flowchart illustrating the processing procedure by the diagnostic ultrasound apparatus 200 according to the embodiment. As illustrated in FIG. 9, the probe position detector 216 detects a position of the probe 201 (Step S101).

Next, the scan plane position calculating circuitry 217 determines a position of the scan plane based on the position of the probe determined by the probe position detector 216 (Step S102). The scan plane position transmitter 218 outputs the position of the scan plane determined by the scan plane position calculating circuitry 217 to the diagnostic X-ray apparatus 300 (Step S103).

The ultrasound image collecting circuitry 213 collects the ultrasound image data (Step S104). The puncture region designating circuitry 219 receives the designation of the region to be punctured from the operator (Step S105). The puncture region designating circuitry 219 then generates the image data that superimposes the region to be punctured on the ultrasound image data (Step S106). The ultrasound image data transmitter 220 outputs the ultrasound image data superimposed by the puncture region designating circuitry 219 to the diagnostic X-ray apparatus 300 (Step S107).

FIG. 10 is a flowchart illustrating the processing procedure by the diagnostic X-ray apparatus 300 according to the embodiment. As illustrated in FIG. 10, the system controller 320 determines whether the position information of the scan plane has been acquired (Step S201). If it is determined that the position information of the scan plane has been acquired (Yes at Step S201), the system controller 320 determines the X-ray irradiation direction (Step S202). The arm movement controller 321 then positions the C-arm 310 at a position calculated by the system controller 320 (Step S203).

The data collecting circuitry 326 determines whether the ultrasound image data has been acquired from the diagnostic ultrasound apparatus 200 (Step S204). If it is determined that the ultrasound image data has been acquired from the diagnostic ultrasound apparatus 200 (Yes at Step S204), the data collecting circuitry 326 creates the projection image data (S205).

Next, the image processing circuitry 327 acquires the X-ray image data from the X-ray image input circuitry 325 (Step S206). The image processing circuitry 327 then generates the superimposed image data that superimposes the projection image data on the X-ray image data (Step S207).

As described above, the puncture supporting system 100 according to the embodiment generates the projection image data that projects the ultrasound image data captured by the diagnostic ultrasound apparatus 200 in the X-ray irradiation direction and generates the superimposed image data superimposed with the X-ray image data captured by the diagnostic X-ray apparatus 300. With this processing, the puncture supporting system 100 can check the position of the puncture needle and the position of the scan plane. With this processing, the puncture supporting system 100 can check whether the puncture needle does not deviate from the scan plane.

The puncture supporting system 100 schematically displays the position of the probe in the superimposed image data, and the operator can ascertain from where the ultrasound waves are transmitted.

The puncture supporting system 100 generates the projection image data projected by applying X-rays in the vertical direction relative to the ultrasound image data captured by the diagnostic ultrasound apparatus 200 and generates the superimposed image superimposed with the X-ray image data captured by the diagnostic X-ray apparatus 300. With this processing, the puncture supporting system 100 can check the position of the puncture needle and the position of the scan plane on which the ultrasound image is projected. With this processing, the puncture supporting system 100 can check whether the puncture needle has reached the region to be punctured.

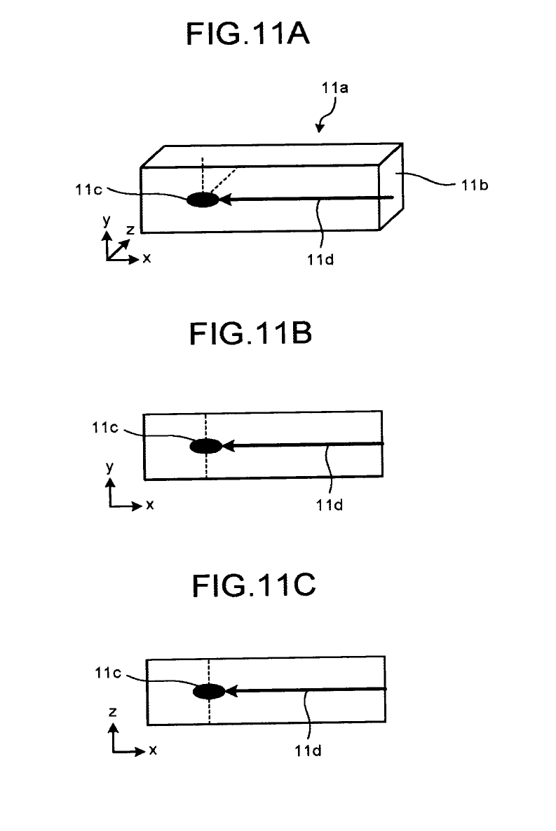

The following describes a relation between the puncture needle and the region to be punctured in the scan plane in the puncture supporting system 100 according to the embodiment with reference to FIG. 11A to FIG. 13C. FIG. 11A to FIG. 13C are diagrams illustrating examples of the relation between the puncture needle and the region to be punctured in the scan plane in the puncture supporting system 100 according to the embodiment.

FIG. 11A is a diagram schematically illustrating a scan plane 11a. The scan plane 11a is illustrated in an x-y-z coordinate system. In FIG. 11A, in the scan plane 11a, a puncture needle 11d is advanced from a contact face 11b with the probe 201 toward a region 11c to be punctured. The tip of the puncture needle lid has the same x-coordinate, y-coordinate, and z-coordinate as those of the region 11c to be punctured. In other words, the tip of the puncture needle 11d has reached the region 11c to be punctured.

FIG. 11B illustrates a projection image when X-rays are applied in the horizontal direction relative to the scan plane 11a illustrated in FIG. 11A to be projected on the x-y plane. As illustrated in FIG. 11B, in the case of the projection in the horizontal direction (the Z-axial direction), the tip of the puncture needle 11d and the region 11c to be punctured are projected in a superimposed manner.

FIG. 11C illustrates a projection image when X-rays are applied in the vertical direction relative to the scan plane 11a illustrated in FIG. 11A to be projected on the z-x plane. As illustrated in FIG. 11C, also in the case of the projection in the vertical direction (the Y-axial direction), the tip of the puncture needle 11d and the region 11c to be punctured are projected in a superimposed manner.

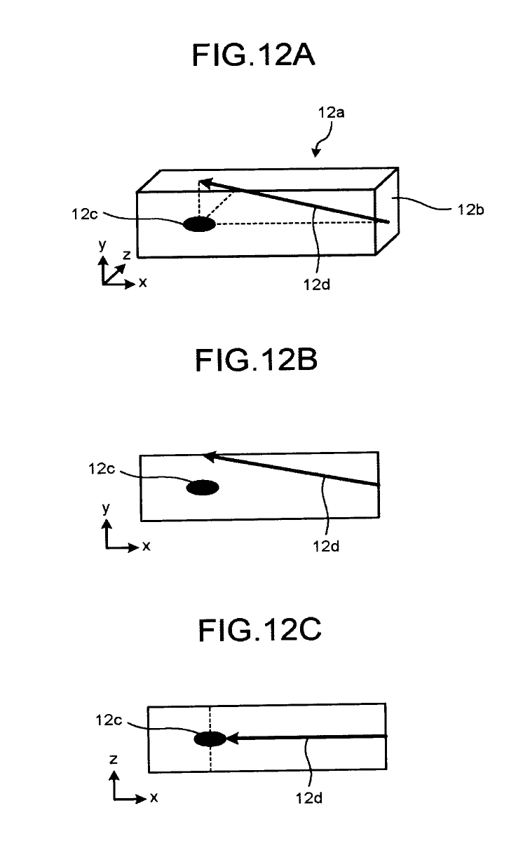

FIG. 12A is a diagram schematically illustrating a scan plane 12a. The scan plane 12a is illustrated in the x-y-z coordinate system. In FIG. 12A, in the scan plane 12a, a puncture needle 12d is advanced from a contact face 12b with the probe 201 toward a region 12c to be punctured. The tip of the puncture needle 12d is positioned at coordinates in which although the x-coordinate and z-coordinate are the same as those of the region 12c to be punctured, the v-coordinate is different from that of the region 12c to be punctured.

FIG. 12B illustrates a projection image when X-rays are applied in the horizontal direction relative to the scan plane 12a illustrated in FIG. 12A to be projected on the x-y plane. As illustrated in FIG. 12B, in the case of the projection in the horizontal direction (the Z-axial direction), information on the y-coordinate is held, and the tip of the puncture needle 11d and the region 11c to be punctured are positioned at different coordinates.

FIG. 12C illustrates a projection image when X-rays are applied in the vertical direction relative to the scan plane 12a illustrated in FIG. 12A to be projected on the z-x plane. As illustrated in FIG. 12C, in the case of the projection in the vertical direction (the Y-axial direction), there is no information on the y-coordinate, and the tip of the puncture needle 11d and the region 11c to be punctured are thus projected in a superimposed manner. In such a case, during the puncture processing, the puncture needle is advanced to the region to be punctured while checking the position of the puncture needle together with the image projected in the horizontal direction, whereby a positional relation between the puncture needle and the region to be punctured can be accurately checked.

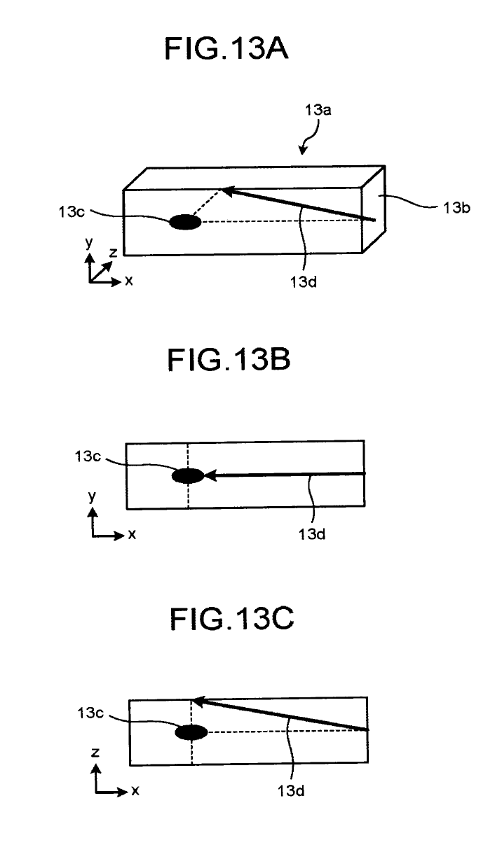

FIG. 13A is a diagram schematically illustrating a scan plane 13a. The scan plane 13a is illustrated in the x-y-z coordinate system. In FIG. 13A, in the scan plane 13a, a puncture needle 13d is advanced from a contact face 13b with the probe 201 toward a region 13c to be punctured. The tip of the puncture needle 13d is positioned at coordinates in which although the x-coordinate and y-coordinate are the same as those of the region 13c to be punctured, the z-coordinate is different from that of the region 13c to be punctured.

FIG. 13B illustrates a projection image when X-rays are applied in the horizontal direction relative to the scan plane 13a illustrated in FIG. 13A to be projected on the x-y plane. As illustrated in FIG. 13B, in the case of the projection in the horizontal direction (the Z-axial direction), there is no information on the z-coordinate, and the tip of the puncture needle 13d and the region 13c to be punctured are thus projected in a superimposed manner.

FIG. 13C illustrates a projection image when X-rays are applied in the vertical direction relative to the scan plane 13a illustrated in FIG. 13A to he projected on the z-x plane. As illustrated in FIG. 13C, in the case of the projection in the vertical direction (the Y-axial direction), information on the z-coordinated is held, and the tip of the puncture needle 13d and the region 13c to be punctured are thus positioned at different coordinates. When X-rays are thus applied in the horizontal direction relative to the scan plane 13a, even when the tip of the puncture needle 13d and the region 13c to be punctured are superimposed on each other, the tip of the puncture needle 13d is actually positioned at coordinates in which although the x-coordinate and y-coordinate are the same as those of the region 13c to be punctured, the z-coordinate is different from that of the region 13c to be punctured. Consequently, the puncture supporting system 100 switches the X-ray irradiation direction relative to the scan plane between the horizontal direction and the vertical direction, whereby the positional relation between the puncture needle and the region to be punctured can be checked more accurately.

Modifications

Although it is described that when X-rays are applied in the horizontal direction relative to the scan plane of the diagnostic ultrasound apparatus 200, the data collecting circuitry 326 generates the projection image data containing the information schematically indicating the scan plane, the information schematically indicating the region to be punctured, and the information schematically indicating the probe, the projection image generated by the data collecting circuitry 326 is not so limited; when X-rays are applied in the horizontal direction relative to the scan plane of the diagnostic ultrasound apparatus 200, the data collecting circuitry 326 may, for example, generate the projection image data containing only the scan plane.

Although it is described that. when X-rays are applied in the vertical direction relative to the scan plane of the diagnostic ultrasound apparatus 200, the data collecting circuitry 326 generates the projection image data containing the ultrasound image, the projection image data may be generated containing information schematically indicating the scan plane in place of the ultrasound image.

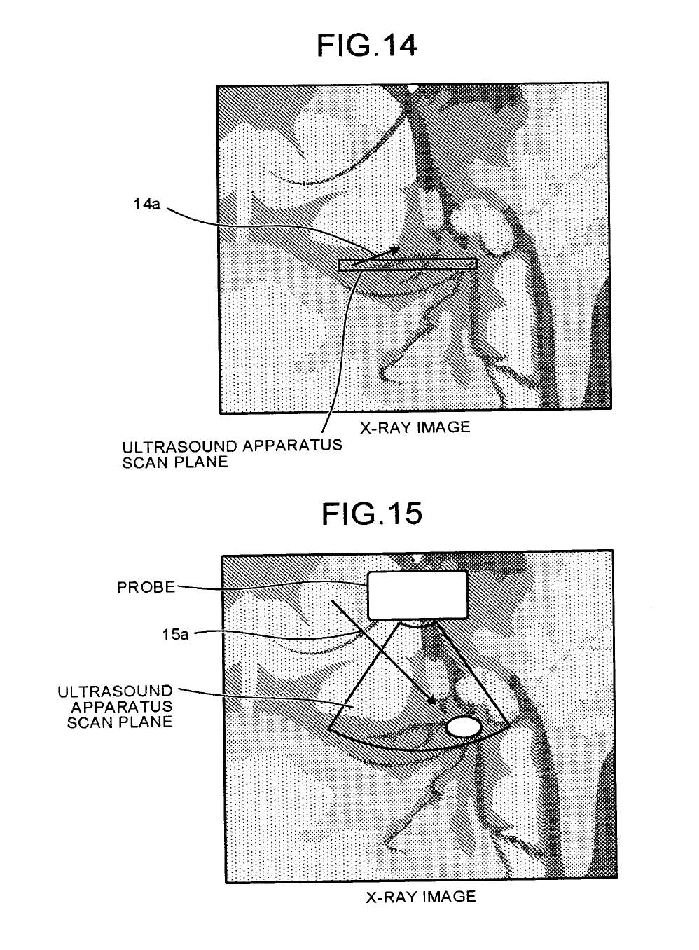

The image processing circuitry 327 generates the superimposed image data using the projection image data generated by the data collecting circuitry 326. Modifications of the superimposed image data generated by the image processing circuitry 327 will be described with reference to FIG. 14 and FIG. 15. Described with reference to FIG. 14 is a modification of the superimposed image data generated when X-rays are applied in the horizontal direction relative to the scan plane of the diagnostic ultrasound apparatus 200. Described with reference to FIG. 15 is a modification of the superimposed image data generated when X-rays are applied in the vertical direction relative to the scan plane of the diagnostic ultrasound apparatus 200. FIG. 14 and FIG. 15 illustrate cases when the pieces of superimposed image data generated by the image processing circuitry 327 are displayed on the display 301.

FIG. 14 is a diagram illustrating an example of the modifications of the superimposed image data generated when X-rays are applied in the horizontal direction relative to the scan plane of the diagnostic ultrasound apparatus 200. As illustrated in FIG. 12A to 12C, the image processing circuitry 327 generates the superimposed image data that superimposes the projection image data on the X-ray image data. Specifically, the image processing circuitry 327 superimposes the projection image data containing only the scan plane of the diagnostic ultrasound apparatus 200 on the X-ray image data. A puncture needle 14a is projected on the X-ray image data. With this processing, the operator can check whether the puncture needle does not deviate from the scan plane of the diagnostic ultrasound apparatus 200.

FIG. 15 is a diagram illustrating an example of the modifications of the superimposed image data generated when X-rays are applied in the vertical direction relative to the scan plane of the diagnostic ultrasound apparatus 200. As illustrated in FIG. 15, the image processing circuitry 327 generates the superimposed image data that superimposes the projection image data on the X-ray image data. Specifically, the image processing circuitry 327 superimposes the projection image data containing the information schematically indicating the scan plane, the information schematically indicating the probe, and the information schematically indicating the region to be punctured on the X-ray image data. A puncture needle 15a is projected on the X-ray image data. With this processing, the operator can check whether the puncture needle has reached the region to be punctured. The image processing circuitry 327 may superimpose the projection image data containing only the scan plane on the X-ray image data.

The data collecting circuitry 326 may generate the projection image data containing, in addition to the information schematically indicating the scan plane, at least one of the information indicating the probe and the information indicating the region to be punctured by the puncture needle.

The above embodiment describes a case of generating the superimposed image data that superimposes the X-ray image data and the projection image data when the X-ray irradiation direction relative to the scan plane is set to the horizontal direction or when the X-ray irradiation direction relative to the scan plane is set to the vertical direction. In such a case, the system controller 320 determines an angle horizontal to the scan plane of the probe or an angle vertical to the scan plane of the probe to be the X-ray irradiation direction. In the thus set. X-ray irradiation direction, the superimposed image data that superimposes the X-ray image data and the projection image data is generated in real time. With this processing, the operator of the diagnostic ultrasound apparatus 200 can check how the puncture needle advances. Consequently, the operator of the diagnostic ultrasound apparatus 200 can check whether the puncture needle does not deviate from the scan plane of the diagnostic ultrasound apparatus.

The system controller 320, for example, determines the X-ray irradiation direction so as to give a set angle in accordance with a change in the position of the probe and controls the C-arm movement controller 321 so as to rotate the C-arm 310 to the determined irradiation direction. In other words, the C-arm follows so that the X-ray irradiation direction is the set angle in accordance with the chance in the position of the probe. With this operation, the operator of the diagnostic ultrasound apparatus 200 can check how the puncture needle advances even when the position of the probe changes.

Although the above embodiment describes a case in which the X-ray irradiation direction relative to the scan plane is the horizontal angle or a case in which the X-ray irradiation direction relative to the scan plane is the vertical angle, the irradiation direction may be changed during the puncture processing. When the irradiation direction is switched during the puncture processing, the operating circuitry 303, for example, further receives an instruction to change the angle from the operator during the processing to perform puncture. The operating circuitry 303, for example, receives the instruction to change the angle from the operator who operates the diagnostic X-ray apparatus 300 in response to a signal by the operator who operates the diagnostic ultrasound apparatus 200 that performs the puncture processing. The system controller 320 determines an angle related to the instruction to change received by the operating circuitry 303 to be a changed X-ray irradiation direction. Next, the data collecting circuitry 326, based on the position information and the changed X-ray irradiation direction, newly generates the projection image data that projects an ultrasound image of the scan plane in the X-ray irradiation direction. The display 301 displays the X-ray image data containing the puncture needle captured in the changed X-ray irradiation direction and the projection image data newly generated by the data collecting circuitry 326. Also when the irradiation direction is thus switched during the puncture processing, the superimposed image data is generated in real time.

Although it is described that when the irradiation direction is switched during the puncture processing, the operator of the diagnostic X-ray apparatus 300 instructs to change the angle in response to the signal by the operator of the diagnostic ultrasound apparatus 200, embodiment are not so limited. The switching of the irradiation direction may be automated, for example. More specifically, the system controller 320 alternately switches the X-ray irradiation angle relative to the scan plane between the horizontal angle and the vertical angle at certain time intervals. The data collecting circuitry 326, based on the position information and the angle-switched X-ray irradiation direction, newly generates the projection image data that projects an ultrasound image of the scan plane in the X-ray irradiation direction. This processing causes the display 301 to display the X-ray image data containing the puncture needle captured in the angle-switched X-ray irradiation direction and the projection image data newly generated by the data collecting circuitry 326. The certain time intervals may be freely set.

Although it is described in the above embodiment that the display 301 displays the superimposed image data that superimposes the X-ray image data and the projection image data generated by the image processing circuitry 327, embodiments are not so limited. The display 301, for example, may display the X-ray image data and the projection image data without being superimposed on each other. In such a case, the image processing circuitry 327, without generating the superimposed image data that superimposes the X-ray image data and the projection image data, sends the X-ray image data and the projection image separately to the image output circuitry 324. The display 301, for example, divides its display area to display the X-ray image data on one display area and display the projection image data on the other display area.

Although the embodiment describes a case in which the diagnostic X-ray apparatus 300 has one C-arm 310, the diagnostic X-ray apparatus 300 may be a diagnostic X-ray apparatus of the biplane type having two C-arms 310. In this case, the diagnostic X-ray apparatus 300 may display the pieces of superimposed image data projected in the horizontal direction and the vertical direction at all times or may display one of the superimposed image data projected in the horizontal direction and the superimposed image data projected in the vertical direction selectively in a switching manner based on instructions by the operator. When the diagnostic X-ray apparatus 300 of the biplane type has a plurality of displays 301, one display 301 may display the superimposed image data projected in the horizontal direction, while the other display 301 may display the superimposed image data projected in the vertical direction. Even for the biplane type diagnostic X-ray apparatus, the display 301 may divide its display area to display the X-ray image data on one display area and display the projection image data on the other display area.

Although the above embodiment describes a case in which the diagnostic X-ray apparatus 300 displays the superimposed image data on the display 301, embodiments are not so limited. The diagnostic ultrasound apparatus 200 may, for example, cause the ultrasound image display 202 to display the superimposed image data. In such a case, the diagnostic X-ray apparatus 300 sends the superimposed image data generated by the image processing circuitry 327 or the X-ray image data and the projection image data to the diagnostic ultrasound apparatus 200. The diagnostic ultrasound apparatus 200 displays the superimposed image data or the X-ray image data and the projection image data received from the diagnostic X-ray apparatus 300 on the ultrasound image display 202. The ultrasound image display 202, when it displays the X-ray image data and the projection image data separately without the X-ray image data and the projection image data superimposed on each other, for example, divides its display area to display the X-ray image data on one display area and display the projection image data on the other display area. The diagnostic X-ray apparatus 300 may send the superimposed image data generated by the image processing circuitry 327, the X-ray image data, and the projection image data to the diagnostic ultrasound apparatus 200. In such a case, the ultrasound image display 202, for example, displays image data selected by the operator of the diagnostic ultrasound apparatus 200 from the superimposed image data or the X-ray image data and the projection image data.

An image processing apparatus that is connected to the puncture supporting system 100 via a network and is different from the diagnostic X-ray apparatus 300 or the diagnostic ultrasound apparatus 200 may receive the superimposed image data or the X-ray image data and the projection image data from the diagnostic X-ray apparatus 300 or the diagnostic ultrasound apparatus 200. In such a case, the image processing apparatus may display the superimposed image data or may display the X-ray image data and the projection image data separately without being superimposed on each other.

The above-described diagnostic X-ray apparatus and diagnostic ultrasound apparatus can check whether the puncture needle does not deviate from the scan plane of the diagnostic ultrasound apparatus.

While certain embodiments have been described, these embodiments have been presented by way of example only, and are riot intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A diagnostic X-ray apparatus comprising:
determining circuitry configured to determine at least one of an angle horizontal to a scan plane of the probe and an angle vertical to the scan plane of the probe to be an X-ray irradiation direction based on position information of a probe of a diagnostic ultrasound apparatus acquired from the diagnostic ultrasound apparatus in processing to perform puncture using a puncture needle;
projection image generating circuitry configured to generate projection image data that projects an ultrasound image of the scan plane in the X-ray irradiation direction based on the position information and the X-ray irradiation direction; and
a display configured to display X-ray image data containing the puncture needle captured in the X-ray irradiation direction and the projection image data generated by the projection image generating circuitry.

2. The diagnostic X-ray apparatus according to claim 1, further comprising operating circuitry configured to receive an instruction to select at least one of the angle horizontal to the scan plane and the angle vertical to the scan plane from an operator, wherein
the determining circuitry determines the angle received by the operating circuitry to be the X-ray irradiation direction.

3. The diagnostic X-ray apparatus according to claim 1, wherein
the operating circuitry further receives an instruction to change the angle from the operator during the processing to perform puncture,
the determining circuitry determines an angle related to the instruction to change received by the operating circuitry to be a changed X-ray irradiation direction,
the projection image generating circuitry newly generates projection image data that projects an ultrasound image of the scan plane in the X-ray irradiation direction based on the position information and the changed X-ray irradiation direction; and
the display displays X-ray image data containing the puncture needle captured in the changed X-ray irradiation direction and the projection image data newly generated by the projection image generating circuitry.

4. The diagnostic X-ray apparatus according to claim 1, wherein the display displays the X-ray image data and the projection image data in a superimposed manner.

5. The diagnostic X-ray apparatus according to claim 1, further comprising:
an arm configured to hold an X-ray source that generates X-rays and an X-ray detector that detects the X-rays from the X-ray source;
operating circuitry configured to perform an operation to control movement of the arm; and
a controller configured to control the movement of the arm so as to give the irradiation direction based on the operation.

6. The diagnostic X-ray apparatus according to claim 1, wherein the projection image generating circuitry generates projection image data containing at least. one of information indicating the probe and information indicating a region to be punctured by the puncture needle.

7. A diagnostic ultrasound apparatus comprising:
an ultrasound probe that transmits and receives ultrasound waves to and from a subject, the ultrasound probe being capable of being fitted with a puncture adapter for fixing the puncture needle in processing to perform puncture using a puncture needle;
image generating circuitry configured to generate image data from reflected waves received from the ultrasound probe;
a controller configured to control transmission and reception of ultrasound waves by the ultrasound probe;
a detector configured to detect position information of the ultrasound probe;
a transmitter configured to transmit the position information of the ultrasound probe and the image data to a diagnostic X-ray apparatus;
a receiver configured to receive X-ray image data and projection image data from the diagnostic X-ray apparatus; and
a display configured to display the X-ray image data containing the puncture needle and the projection image data, wherein
the X-ray image data containing the puncture needle is captured by the diagnostic X-ray apparatus in an X-ray irradiation direction determined to be at least one of an angle horizontal to a scan plane of the ultrasound probe and an angle vertical to the scan plane of the ultrasound probe based on the position information of the ultrasound probe; and
the projection image data is generated by projecting an ultrasound image of the scan plane in the X-ray irradiation direction based on the position information and the X-ray irradiation direction.
